(19) United States
(12) Patent Application Publication
(10) Pub. No.: US 2026/0092021 A1
(43) Pub. Date: Apr. 2, 2026

(54) A METHOD OF FORMING A LIQUID HYDROCARBON PRODUCT

(71) Applicant: JOHNSON MATTHEY DAVY TECHNOLOGIES LIMITED, London (GB)

(72) Inventors: Robert Miles BAKER, London (GB); Simon HILL, London (GB)

(21) Appl. No.: 19/113,152

(22) PCT Filed: Oct. 20, 2023
(86) PCT No.: PCT/GB2023/052729

§ 371 Date: Mar. 19, 2025

(30) Foreign Application Priority Data
Nov. 11, 2022 (GB) 2216857.9

(51) Int. Cl. C07C 1/10

(52) U.S. Cl. CPC C07C 1/10

(57) ABSTRACT

A method of forming a liquid hydrocarbon product comprising: providing a feed gas of carbon, hydrogen and oxygen to generate a syngas, the syngas comprising carbon monoxide, hydrogen and steam; cooling the syngas to form an aqueous condensate and a water-depleted syngas, the aqueous condensate having a carbon-containing gas dissolved therein; stripping the aqueous condensate with steam to transfer carbon-containing gas from the aqueous condensate to the steam to thereby form a stripped aqueous condensate and a first stripper effluent steam; passing the water-depleted syngas to a Fischer-Tropsch unit to form a liquid hydrocarbon product and a co-produced water, the co-produced water having carbon-containing substances dissolved therein; stripping the co-produced water to transfer carbon-containing substances to the first stripper effluent steam to thereby form a stripped co-produced water and a second stripper effluent steam; and recycling the second stripper effluent steam into the feed gas.

### FIELD

The invention relates to a method of forming a liquid hydrocarbon product.

### BACKGROUND

The Fischer-Tropsch process uses chemical reactions to convert a mixture of carbon monoxide and hydrogen into liquid hydrocarbons. These reactions occur in the presence of metal catalysts, typically at temperatures of 150-300° C. and pressures of one to several tens of atmospheres. The Fischer-Tropsch process produces a variety of hydrocarbons, ideally having the formula (CₙH₂ₙ₊₂). The more useful reactions produce alkanes as follows:

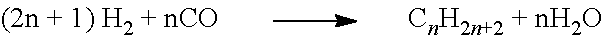

where n is typically 1 to 100. The formation of methane (n=1) is unwanted. In addition to alkane formation, competing reactions give small amounts of alkenes, as well as alcohols and other oxygenated hydrocarbons. The Fischer-Tropsch reaction is a highly exothermic reaction due to a standard reaction enthalpy (ΔH) of −165 KJ/mol CO combined.

Synthesis gas (syngas) feed to a Fischer-Tropsch unit can be derived from a number of feedstocks; for example, natural gas via steam reforming and/or auto-thermal reforming, municipal solid waste and biomass via high-temperature gasification, or carbon dioxide and hydrogen via a reverse-water-gas-shift reaction. The latter source is beneficial since it makes use of carbon dioxide that may have been destined to be released to the atmosphere.

WO2022/079408A1 discloses use of an autothermal reverse-water-gas-shift unit in a hydrocarbon synthesis process. A process condensate is recovered from a reverse-water-gas-shift unit and a co-produced water is recovered from a hydrocarbon synthesis (Fischer-Tropsch) unit. The process condensate and co-produced water are separately stripped to reduce their organic contaminates, reduce the burden on downstream water treatment, and return carbon back to the process. Such a process requires a large volume of steam. As a result, the energy requirements and water requirements of the process are high. In addition, the water contents of the stripping effluents, at least from the stripped process condensate, are high meaning that not all of the stripping effluents may be recycled back into the process. This results in a penalty to the carbon efficiency of the process. Different examples of stripping condensate or Fischer-Tropsch water are disclosed in WO2021175785A1 and WO2021185865A1, respectively.

The present invention seeks to tackle at least some of the problems associated with the prior art or at least to provide a commercially acceptable alternative solution thereto.

### SUMMARY

The present disclosure is directed to a method of forming a liquid hydrocarbon product, the method comprising:
providing a feed gas comprising compounds of the elements carbon, hydrogen and oxygen;
generating a syngas from the feed gas, the syngas comprising carbon monoxide, hydrogen and steam;
cooling the syngas to below the dew point to form an aqueous condensate and a water-depleted syngas, the aqueous condensate having a carbon-containing gas dissolved therein;
passing the aqueous condensate to a first stripper and stripping the aqueous condensate with steam to transfer carbon-containing gas from the aqueous condensate to the steam to thereby form a stripped aqueous condensate and a first stripper effluent steam;
passing the water-depleted syngas to a Fischer-Tropsch unit to form a liquid hydrocarbon product and a co-produced water, the co-produced water having carbon-containing substances dissolved therein;
passing the co-produced water to a second stripper and stripping the co-produced water with the first stripper effluent steam to form a stripped co-produced water and a second stripper effluent steam; and
recycling the second stripper effluent steam into the feed gas.

### BRIEF DESCRIPTION OF THE DRAWINGS

FIG. 1 depicts a flow diagram of one embodiment of a method according to the present invention.

FIG. 2 depicts a flow diagram of a further embodiment of a method according to the present invention.

### DETAILED DESCRIPTION

In a first aspect, the present disclosure is directed to a method of forming a liquid hydrocarbon product, the method comprising:
providing a feed gas comprising compounds of the elements carbon, hydrogen and oxygen;
generating a syngas from the feed gas, the syngas comprising carbon monoxide, hydrogen and steam;
cooling the syngas to below the dew point to form an aqueous condensate and a water-depleted syngas, the aqueous condensate having a carbon-containing gas dissolved therein;
passing the aqueous condensate to a first stripper and stripping the aqueous condensate with steam to transfer carbon-containing gas from the aqueous condensate to the steam to thereby form a stripped aqueous condensate and a first stripper effluent steam;
passing the water-depleted syngas to a Fischer-Tropsch unit to form a liquid hydrocarbon product and a co-produced water, the co-produced water having carbon-containing substances dissolved therein;
passing the co-produced water to a second stripper and stripping the co-produced water with the first stripper effluent steam to transfer carbon-containing substances from the co-produced water to the first stripper effluent steam to thereby form a stripped co-produced water and a second stripper effluent steam; and
recycling the second stripper effluent steam into the feed gas.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

Advantageously, recycling the second stripper effluent steam into the feed gas may increase the carbon efficiency of the method. In contrast to the aqueous condensate and co-produced water, the second stripper effluent steam is in the form of a gas, typically a high-pressure gas, meaning that it can be re-introduced into the feed gas without requiring any significant prior heating and/or pressurisation steps. As a result, the energy efficiency of the method may be increased.

The aqueous condensate, by virtue of being derived from a syngas generation step, tends to be relatively “clean” or pure in comparison to the co-produced water formed in the Fischer-Tropsch unit. In other words, it tends to contain lower levels of carbon-containing gases and/or carbon-containing substances. This means that the first stripper effluent steam may be suitable for use in stripping the “dirtier” or more heavily contaminated co-produced water. By using the first stripper effluent steam to strip the co-produced water, the method requires less steam in comparison to conventional methods in which the aqueous condensate and co-produced water are stripped in separate stripper units, i.e., in parallel. Accordingly, in comparison to conventional methods, the water requirements and the energy requirements of the method may be reduced. Furthermore, in conventional methods employing “parallel” stripping, the aqueous stripper effluent steam may have a relatively low concentration of organics and a relatively high concentration of steam. This may mean that the aqueous stripper effluent steam is unsuitable for recycling to the feed gas, particularly without energy-intensive further treatments steps, and must instead be disposed of, thereby incurring waste treatment costs and resulting in a loss of organics or carbon compounds from the process. In other words, the method of the present invention may enable recycling of substantially all organics contained in both of the stripper effluent steams.

In comparison to alternative methods in which the aqueous condensate and co-produced water are combined and then stripped in a single stripper unit, the method of the present invention may result in a final stripper effluent steam (i.e., the second stripper effluent steam) that is more concentrated in organics. This may render it more suitable to be recycled into the feed gas for use in the syngas generation step. In addition, the method of the present invention may result in two stripped products, i.e., the stripped aqueous condensate and the stripped co-produced water. This is beneficial since the stripped aqueous condensate derived from the syngas generation step is likely to be “cleaner” (have a lower chemical oxygen demand, COD) than the stripped co-produced water derived from the Fischer-Tropsch reaction. This means that the stripped aqueous condensate may be treated more easily. As a result, the overall water-treatment burden of the method may be reduced.

The method forms a liquid hydrocarbon product. The term “liquid hydrocarbon” as used herein may encompass species formed of carbon and hydrogen that are liquid at room temperature and pressure. The hydrocarbons typically comprise alkanes, and typically comprise from 5 to 30 carbon atoms per molecule. The method preferably comprises recovering the liquid hydrocarbon product.

The method comprises providing a feed gas comprising compounds of the elements carbon, hydrogen and oxygen. For the avoidance of doubt, the feed gas contains each of the elements carbon, hydrogen and oxygen. However, the compounds contained in the feed gas may contain only one, only two or all three of these elements. For example, the feed gas may comprise carbon dioxide (CO₂) and hydrogen (H₂), and/or methane (CH₄) and steam (H₂O).

The method comprises generating a syngas from the feed gas, the syngas comprising carbon monoxide (CO), hydrogen (H₂) and steam (H₂O). As discussed in more detail below, generating the syngas may comprise, for example, a reverse-water-gas-shift reaction and/or a steam reforming reaction and/or a partial oxidation reaction.

The term “reverse-water-gas-shift reaction” as used herein may encompass the reaction of carbon dioxide and hydrogen to form carbon monoxide and steam, i.e.

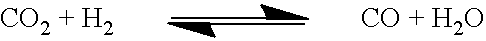

The term “steam reforming reaction” as used herein may encompass the reaction of methane and steam to form carbon monoxide and hydrogen, i.e.,

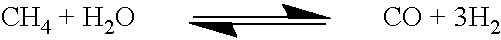

The term “partial oxidation” as used herein may encompass the reaction of methane and molecular oxygen to form carbon monoxide and hydrogen, i.e.,

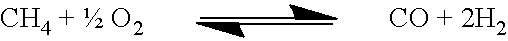

The method of the present invention comprises cooling the syngas to below the dew point to form an aqueous condensate and a water-depleted syngas. The term “dew point” as used herein may encompass the temperature at which a gas must be cooled in order to become saturated with water vapour assuming constant pressure and temperature. Cooling to below the dew point causes aqueous condensate to form. The aqueous condensate has a carbon-containing gas (i.e., a gas of one or more carbon-containing compounds) dissolved therein. Such carbon-containing gas may comprise, for example, one or more of carbon monoxide, carbon dioxide and methane.

The method comprises passing the aqueous condensate to a first stripper and stripping the aqueous condensate with steam. The term “stripping” as used herein may encompass the physical separation process where one or more components are removed from a liquid stream by a vapor stream. Suitable stripping apparatus are known in the art. The stripping is typically carried out in a packed or trayed column, but may also be carried out in, for example, a spray tower, a bubble column and/or a centrifugal contactor. The stripping is typically carried out at elevated temperature and/or pressure. Suitable elevated temperatures and pressures are known in the art. The stripping may transfer carbon-containing gas from the aqueous condensate to the steam to thereby form a stripped aqueous condensate and a first stripper effluent steam. The stripping typically transfers the majority of the carbon-containing gas from the aqueous condensate to the steam, more typically at least 50% by mass of the carbon-containing gas, more typically at least 75% by mass, even more typically at least 90% by mass, even more typically at least 95% by mass, even more typically substantially all of the carbon-containing gas.

The method further comprises passing the water-depleted syngas to a Fischer-Tropsch unit to form a liquid hydrocarbon product and a co-produced water. Fischer-Tropsch units are known in the art. The Fischer-Tropsch process uses a collection of chemical reactions to convert a mixture of carbon monoxide and hydrogen into liquid hydrocarbons. These reactions occur in the presence of metal catalysts, typically at temperatures of 150-300° C. and pressures of one to several tens of atmospheres. The Fischer-Tropsch process produces a variety of hydrocarbons, ideally having the formula (CₙH₂ₙ₊₂). The more useful reactions produce alkanes as follows:

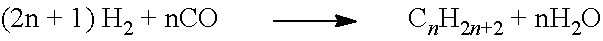

where n is typically 1-100.

The co-produced water formed as a result of the Fischer-Tropsch reactions has carbon-containing substances (carbon-containing compounds) dissolved therein. Such carbon-containing substances may include, for example, one or more of carbon monoxide, carbon dioxide, methane, alcohols and carboxylic acids.

The method further comprises passing the co-produced water to a second stripper and stripping the co-produced water with the first stripper effluent steam to transfer carbon-containing substances from the co-produced water to the first stripper effluent steam to thereby form a stripped co-produced water and a second stripper effluent steam. As discussed above, suitable stripper apparatus are known in the art. The first and second strippers may be the same or different. The stripping is typically carried out at elevated temperature and/or pressure. The stripping typically transfers the majority of the carbon-containing substances from the aqueous condensate to the steam, more typically at least 50% by mass of the carbon-containing substances, more typically at least 75% by mass, even more typically at least 90% by mass, even more typically at least 95% by mass, even more typically substantially all of the carbon-containing substances.

The method further comprises recycling the second stripper effluent steam into the feed gas. As discussed above, by virtue of being a high-pressure gas, the second stripper effluent steam may be incorporated into the feed gas without any substantial pressurisation and/or heating steps. Carbon-containing substances in the second stripper effluent steam, such as one or more of carbon monoxide, carbon dioxide, methane, alcohols and carboxylic acids, may be converted to syngas.

Generating a syngas from the feed gas preferably comprises converting at least a portion of the feed gas to carbon monoxide. Usefully, reactions for the conversion of the feed gas to carbon monoxide use steam. Use of steam in these reactions advantageously reduces catalyst deactivation.

Preferably, the feed gas comprises carbon dioxide and hydrogen; and converting at least a portion of the feed gas to carbon monoxide comprises subjecting the feed gas to a reverse-water-gas-shift reaction. A reverse-water-gas-shift reaction is particularly suitable for converting at least a portion of the feed gas to carbon monoxide. At least some of the hydrogen may remain in the syngas, i.e., it is not converted to water in the reverse-water-gas-shift reaction. This enables it to be used in the subsequent Fischer-Tropsch reaction. Since the reverse-water-gas shift reaction uses carbon dioxide, it can make use of carbon dioxide produced by combustion that might otherwise be vented to the atmosphere.

The reverse-water-gas-shift reaction is preferably an autothermal reverse-water-gas-shift reaction, an electrically heated reverse-water-gas-shift reaction or a plasma-heated reverse-water-gas-shift reaction. Such reactions are particularly suitable.

The reverse-water-gas-shift reaction is preferably carried out using a catalyst comprising nickel, more preferably wherein the catalyst comprises from 3 to 20 wt. % nickel, expressed as NiO, on a refractory metal oxide support, based on the total weight of the reverse-water-gas-shift catalyst. Such catalysts may enable the reverse-water-gas-shift reaction to be carried out at reduced temperatures and/or pressures and/or with a high yield and/or with a high selectivity. Moreover, such catalysts are active for the conversion by steam reforming of the carbon-containing substances in the second stripper effluent steam into syngas.

The reverse-water-gas-shift reaction is preferably carried out at a temperature of at least 700° C. Such temperatures may result in a particularly high yield.

In alternative arrangements, the feed gas comprises methane and steam; and converting at least a portion of the feed gas to carbon monoxide comprises subjecting the feed gas to a steam reforming reaction. A steam reforming reaction is particularly suitable for converting at least a portion of the feed gas to carbon monoxide.

The steam reforming reaction is preferably carried out using a catalyst comprising nickel.

The steam reforming is preferably carried out under a pressure of from 15 to 55 bara and/or a temperature of from 750 to 1100° C. Such conditions may result in a particularly high yield and/or selectivity.

The steam reforming reaction preferably comprises one or more stages of adiabatic steam reforming, fired steam reforming, gas-heated reforming, electrically heated reforming and autothermal steam reforming.

Preferably, the ratio by mass (or moles) of steam to aqueous condensate in the first stripper is from 0.1:1 to 0.5:1, more preferably from 0.2:1 to 0.4:1, even more preferably from 0.25:1 to 0.35:1, still even more preferably about 0.3:1. Lower levels of steam may result in less efficient stripping. Higher levels of steam may increase the cost of the method without any significant increase in stripping efficiency.

By operating the first stripper within the above ranges, the ratio by mass (or moles) of first stripper effluent steam to co-produced water in the second stripper may be from 0.2:1 to 0.6:1, preferably from 0.3:1 to 0.5:1, more preferably from 0.35:1 to 0.45:1, even more preferably about 0.4:1. Lower levels of first stripper effluent steam may result in less efficient stripping. Higher levels of first stripper effluent steam may increase the cost of the method without any significant increase in stripping efficiency.

The first stripper preferably operates at a higher pressure than the second stripper. The first stripper more preferably operates at a pressure 100 to 200 kPa higher than the pressure of the second stripper. This may make it easier to pass the first stripper effluent steam into the second stripper due to the pressure drop that may occur in the first stripper.

The first stripper and the second stripper preferably operate at a pressure from 1500 to 5500 kPa. Such pressures may be particularly suitable for transferring the carbon-containing gas from the aqueous condensate to the steam and/or for transferring carbon-containing substances from the co-produced water to the first stripper effluent steam. In addition, the use of such pressures results in the second stripper effluent steam being at a pressure suitable for incorporation into the feed gas without having to undergo a substantial pressurisation step, which would reduce the energy efficiency of the method. Lower pressures, such as from 1500 to 3000 kPa, are preferably employed when the syngas is generated using a reverse-water-gas-shift reaction. Higher pressures, such as from 3000 to 5500 kPa, are preferably employed when the syngas is generated using a steam reforming reaction.

The method may further comprise passing the stripped aqueous condensate to a demineralised water plant to produce water for producing steam. Since the stripped aqueous condensate tends to be relatively “clean” it may be passed to a demineralised water plant without any substantial pre-treatment steps. The produced steam is preferably used in the first stripper or for heat exchange with components of the feed gas or for heat exchange within the Fischer-Tropsch unit.

The method may further comprise passing the stripped co-produced water to a water-treatment unit to produce a water effluent stream. Such a water effluent stream may be “clean” enough for subsequent disposal.

The carbon-containing gas dissolved in the aqueous condensate and/or the carbon-containing substances dissolved in the co-produced water preferably comprises carbon dioxide and/or carbon monoxide and/or organic compounds (e.g., water-soluble organic compounds). It may be beneficial to recycle such species in the second stripper effluent steam since such species may be employed in the syngas generation step. For example, such species are typically converted in a reverse-water-gas-shift unit or a steam reformer unit into synthesis gas.

The molar ratio of hydrogen to carbon monoxide in the water-depleted syngas is preferably from 1.8 to 2.2. Such a ratio is particularly suitable for the subsequent Fischer-Tropsch reaction.

The Fischer-Tropsch unit preferably operates at a temperature of from 150° C. to 300° C. Lower temperatures may result in unfavourably low levels of liquid hydrocarbons being generated. Higher temperatures may increase the energy cost of the method without a significant increase in the levels of liquid hydrocarbons being produced and can increase unwanted by-product formation.

The Fischer-Tropsch unit preferably comprises a catalyst comprising cobalt, iron and/or ruthenium, preferably cobalt. Such a catalyst may be particularly effective at catalysing Fischer-Tropsch reactions and/or enable the reaction to proceed favourably to alkanes at low temperatures and/or with high yield.

The liquid hydrocarbon product preferably comprises alkanes. Alkanes may be a particularly desirable product.

One or more of the compounds in the feed gas may be derived from gasification of biomass and/or municipal waste.

In a preferred embodiment, the second stripper effluent steam may be recycled directly into the feed gas. In other words, the second stripper effluent steam may be recycled into the feed gas without any substantial further treatment steps. In an alternative preferred embodiment, prior to recycling the second stripper effluent steam into the feed gas, the second stripper effluent steam may be passed to a derichment reactor to convert hydrocarbons having two or more carbon atoms contained in the second stripper effluent steam to methane. Methane may be more easily converted to carbon monoxide in the syngas generation step.

The invention will now be described with reference to the Figures. It will be understood by those skilled in the art that the drawings are diagrammatic and that further items of equipment such as feedstock drums, pumps, vacuum pumps, compressors, gas recycling compressors, temperature sensors, pressure sensors, pressure relief valves, control valves, flow controllers, level controllers, holding tanks, storage tanks and the like may be required in a commercial plant.

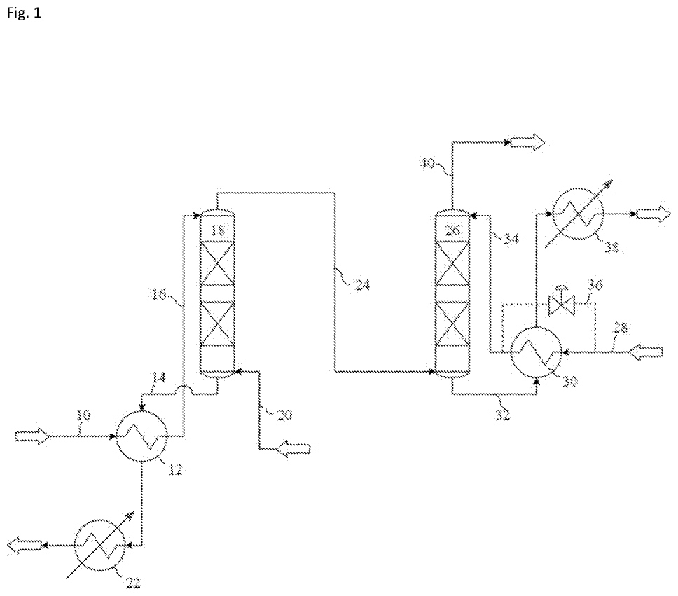

Referring to FIG. 1, an aqueous process condensate stream 10 recovered from a synthesis gas generation unit (not shown) is first heated by interchange with process condensate stripper bottoms from line 14 in interchanger 12 and fed via line 16 to near the top of a first stripper 18. The first stripper contains structured packing to enhance the removal of dissolved gases from the aqueous process condensate. The heated process condensate passes downwards through the first stripper 18 where it contacts with high-pressure steam fed via line 20 to near the bottom of the first stripper. The high-pressure steam strips carbon-containing gases from the heated process condensate. The stripped liquid process condensate stream 14 recovered from the bottom of the first stripper 18 is first cooled by interchanging with the first stripper feed in interchanger 12 and subsequently against cooling water or air cooling in one or more further heat exchangers 22, for export to a water treatment unit (not shown). The stripped aqueous condensate may be fed to a demineralised water unit for purification and use in the process. A first stripper effluent steam is recovered from the fop of the first stripper 18 and fed via line 24 to near the bottom of a second stripper 26. A Fischer-Tropsch co-produced water stream from a Fischer-Tropsch synthesis unit (not shown) fed by line 28 is first heated by interchange with second stripper bottoms from line 32 in an interchanger 30 and fed via line 34 to near the top of the second stripper 26. A bypass 36 around interchanger 30 is used to control the inlet temperature to the second stripper 26. The second stripper contains structured packing to enhance the removal of dissolved substances from the co-produced water. The heated co-produced water passes downwards through the second stripper 26 where it contacts with the first stripper effluent steam fed via line 24. The first stripper effluent steam strips carbon-containing substances from the co-produced water. The stripped co-produced water stream 32 is first cooled by interchanging with the second stripper feed in interchanger 30 and subsequently against cooling water or air cooling in one or more further heat exchangers 38 for export to a water treatment unit (not shown) for further purification. A second stripper effluent steam containing carbon-containing substances is recovered from the top of the second stripper 26 and fed via line 40 to the syngas generation unit (not shown) for recycling into a syngas generation unit feed gas.

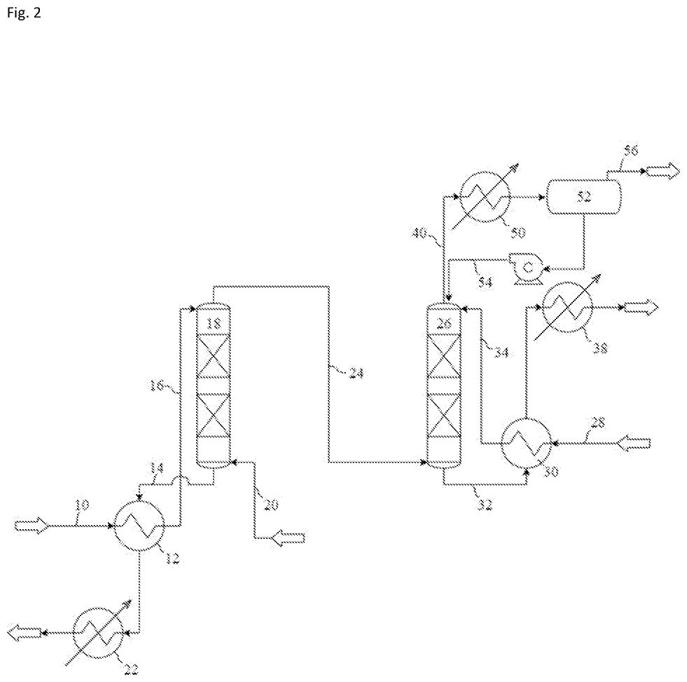

With reference to FIG. 2, there is shown a similar embodiment to that of FIG. 1. However, in the embodiment of FIG. 2 the bypass 36 is omitted and a heat exchanger 50 and gas-liquid separator 52 are used to condense a portion of the second stripper effluent steam and separate a stripper effluent condensate, which is returned via a pump to near the top of the second stripper 26 via line 54. The remaining second stripper effluent steam is recovered from the gas-liquid separator 52 and fed via line 56 to the syngas generation unit (not shown) for recycling into the syngas generation unit feed gas.

The process according to the invention allows a higher recovery of carbon-containing substances from the aqueous streams than alternative methods using a single stripper or two strippers operating in parallel.

The invention will now be described in relation to the following non-limiting example and comparative example.

### Example

A process according to FIG. 1, was designed to provide a stream of steam to a derichment vessel in an upstream syngas generation unit. In the syngas generation unit, the derichment vessel provides a feed comprising carbon dioxide, hydrogen and methane to an autothermal reverse-water-gas-shift reactor (rWGS unit) operating at about 30 bara, that, after aqueous condensate separation, provides a synthesis gas comprising carbon monoxide and hydrogen for use in a Fischer-Tropsch synthesis reaction to produce liquid hydrocarbons. The Fischer-Tropsch reaction produces a stream of co-produced water as a by-product.

The steam-to-aqueous-condensate ratio used for the first stripper was 0.3:1. The Applicant has found it beneficial to maximise the steam carryover from the first stripper as this both reduces the chemical oxygen demand (COD) of the stripped aqueous condensate, thereby reducing the water treatment requirements, and has a diluting effect on the contaminants in the stripped Fischer-Tropsch co-produced water stream. This may be achieved by maximising the amount of heat transferred in the interchanger 12 by using a small hot-end temperature approach, for example about 35° C. The exit temperature from interchanger 30 which sets the co-produced water temperature into the second stripper was adjusted such that the overheads rate from the second stripper was equal to about 90% of the steam addition required upstream of the derichment vessel. This allowed for 10% direct steam addition to the derichment vessel for additional controllability. A typical specification for the raw aqueous condensate stream is:

Raw aqueous condensate specification

| | ppmwt |
|---|---|
| COD | 60 |
| Total carbon species as atomic C | 600 |

A typical specification for the raw co-produced water stream is:

Raw co-produced water specification

| | ppmwt |
|---|---|
| COD | 39000 |
| Total carbon species as atomic C | 10200 |

Using high-pressure steam provided by the rWGS unit at 50 bara saturated steam, a steam-to-condensate ratio into the first stripper of 0.3:1 and an inlet aqueous condensate temperature of 200° C. (equivalent to 35° C. approach in interchanger) results in an inlet temperature of co-produced water to the second stripper of 145° C. (equivalent to an approach of 90° C.) in order to obtain a suitable amount of steam for the derichment vessel. This results in the following in relation to the aqueous condensate, the co-produced water and process carbon efficiency (compared to not recovering any carbon from the condensate streams via stripping):

| | |
|---|---|
| Reduction in aqueous | 95% |
| condensate COD | |
| Increase in aqueous condensate | 9% |
| flow | |
| Reduction in co-produced water | 86% |
| COD | |
| Increase in co-produced water | 21% |
| flow | |
| Improvement in carbon | 1% |
| efficiency | |

### Comparative Example

By means of comparison, to illustrate the improvement of the invention in allowing integration with the same rWGS unit, the stripping could instead be carried out in two stripping columns in parallel. Setting the approach in both interchangers to 35° C. to minimise steam addition and adjusting the steam-to-condensate ratio of each column to achieve similar COD reduction results in (compared to not recovering any carbon from the condensate streams via stripping):

| | |
|---|---|
| Reduction in aqueous condensate | 95% |
| COD | |
| Increase in aqueous condensate | 8% |
| flow | |
| Reduction in co-produced water | 85% |
| COD | |
| Increase in co-produced water | 7% |
| flow | |
| Improvement in carbon | 0.9% |
| efficiency | |
| Stripping steam condensed (as % | 45% |
| of co-produced water flow) | |

Most of the organic compounds are contained in the co-produced water stripper overheads, so the penalty on carbon efficiency is only slight. However, the total overheads steam produced from both strippers is 370% of the process requirement, meaning the remainder of the steam would need to be condensed and sent to water treatment. Hence, the steam, energy and carbon use is much less efficient than the present invention. Furthermore, the two-stage series arrangement effectively allows the same steam to be used for both stripping columns given the stripping duty of the first column is much lighter than the second.

The foregoing detailed description has been provided by way of explanation and illustration and is not intended to limit the scope of the appended claims. Many variations in the presently preferred embodiments illustrated herein will be apparent to one of ordinary skill in the art and remain within the scope of the appended claims and their equivalents.

1-24. (canceled)

25. A method of forming a liquid hydrocarbon product, the method comprising:
providing a feed gas comprising compounds of the elements carbon, hydrogen and oxygen;
generating a syngas from the feed gas, the syngas comprising carbon monoxide, hydrogen and steam;
cooling the syngas to below the dew point to form an aqueous condensate and a water-depleted syngas, the aqueous condensate having a carbon-containing gas dissolved therein;
passing the aqueous condensate to a first stripper and stripping the aqueous condensate with steam to transfer the carbon-containing gas from the aqueous condensate to the steam to thereby form a stripped aqueous condensate and a first stripper effluent steam;
passing the water-depleted syngas to a Fischer-Tropsch unit to form a liquid hydrocarbon product and a co-produced water, the co-produced water having carbon-containing substances dissolved therein;
passing the co-produced water to a second stripper and stripping the co-produced water with the first stripper effluent steam to transfer carbon-containing substances from the co-produced water to the first stripper effluent steam to thereby form a stripped co-produced water and a second stripper effluent steam; and,
recycling the second stripper effluent steam into the feed gas.

26. The method of claim 25, wherein generating a syngas from the feed gas comprises converting at least a portion of the feed gas to carbon monoxide.

27. The method of claim 26, wherein:
the feed gas comprises carbon dioxide and hydrogen; and
converting at least a portion of the feed gas to carbon monoxide comprises subjecting the feed gas to a reverse-water-gas-shift reaction.

28. The method of claim 26, wherein:
the feed gas comprises methane and steam; and
converting at least a portion of the feed gas to carbon monoxide comprises subjecting the feed gas to a steam reforming reaction.

29. The method of claim 25, wherein the ratio by mass of steam to aqueous condensate in the first stripper is from 0.1:1 to 0.5:1, preferably from 0.2:1 to 0.4:1, more preferably from 0.25:1 to 0.35:1, even more preferably about 0.3:1.

30. The method of claim 25, wherein the ratio by mass of first stripper effluent to co-produced water in the second stripper is from 0.2:1 to 0.6:1, preferably from 0.3:1 to 0.5:1, more preferably from 0.35:1 to 0.45:1, even more preferably about 0.4:1.

31. The method of claim 25, wherein the first stripper operates at a higher pressure than the second stripper.

32. The method of claim 31, wherein the first stripper operates at a pressure 100 to 200 kPa higher than the pressure of the second stripper.

33. The method of claim 25, wherein the first stripper and the second stripper operate at a pressure of from 1500 to 5500 kPa.

34. The method of claim 25, further comprising passing the stripped aqueous condensate to a demineralised water plant to produce water for producing steam, preferably wherein the steam is used in the first stripper.

35. The method of claim 25, further comprising passing the stripped co-produced water to a water-treatment unit to produce a water effluent stream.

36. The method of claim 25, wherein the carbon-containing gas dissolved in the aqueous condensate and/or the carbon-containing substances dissolved in the co-produced water comprises carbon dioxide and/or carbon monoxide and/or water-soluble organic compounds.

37. The method of claim 25, wherein the molar ratio of hydrogen to carbon monoxide in the water-depleted syngas is from 1.8 to 2.2.

38. The method of claim 25, wherein, prior to recycling the second stripper effluent steam into the feed gas, the second stripper effluent steam is passed to a derichment reactor to convert hydrocarbons contained in the second stripper effluent steam to methane.
